(19) United States
(12) Patent Application Publication
(10) Pub. No.: US 2020/0289034 A1
(43) Pub. Date: Sep. 17, 2020

(54) CONTROL DEVICE OF MOTION SUPPORT DEVICE

(71) Applicant: Honda Motor Co.,Ltd., Tokyo (JP)

(72) Inventor: Taizo YOSHIKAWA, Saitama (JP)

(73) Assignee: Honda Motor Co.,Ltd., Tokyo (JP)

(21) Appl. No.: 16/807,186

(22) Filed: Mar. 3, 2020

(30) Foreign Application Priority Data
Mar. 11, 2019 (JP) 2019-043699

(51) Int. Cl. A61B 5/11; A61B 5/107; A61B 5/00; A61H 3/00

(52) U.S. Cl. CPC A61B 5/1116; A61B 5/112; A61B 5/1123; A61B 5/1071; A61B 5/6812; A61H 2201/5064; A61B 5/6829; A61H 3/00; A61H 2201/164; A61H 2201/165; A61B 5/6828

(57) ABSTRACT

A control device of a motion support device that can quickly and appropriately support a predetermined motion of a user wearing the motion support device when the user performs the predetermined motion is provided. A control device estimates whether a user is in a standing state, calculates an x-axis speed V_Wx of a waist part of the user M and the absolute value VA_Wx thereof, estimates that the user has started a crouching motion from the standing state if V_Wx<0 and VA_Wx>Vjud1, and controls the walking assist device such that the crouching motion is supported if the user is estimated to have started the crouching motion.

### CROSS-REFERENCE TO RELATED APPLICATION

This application claims the priority of Japan patent application serial no. 2019-043699, filed on Mar. 11, 2019. The entirety of the above-mentioned patent application is hereby incorporated by reference herein and made a part of this specification.

### BACKGROUND

### Technical Field

The disclosure relates to a control device of a motion support device that supports a predetermined motion of a user.

### Description of Related Art

A motion estimation method disclosed in Patent Document 1 (Japanese Laid-open No. 2018-15023) is known in the related art. In this motion estimation method, a user wears one mobile terminal on his or her thigh, and a motion of the user is estimated on the basis of a detection signal of an acceleration sensor installed in the mobile terminal. Specifically, accelerations in directions of three axes (directions of x, y, and z axes) are calculated on the basis of detection signals of the acceleration sensor, and whether a user has transitioned a posture from a “standing posture” or one of a “sitting posture” and a “crouching posture” is estimated on the basis of change in a magnitude relation of the absolute values of the accelerations.

According to the motion estimation method of the related art, one acceleration sensor is used to estimate transition of a motion of the user. However, according to the related art, it is not possible to estimate the start of the motion as long as a posture of the user does not significant1y change and the magnitude relation of the absolute values of accelerations in the directions of the three axes does not significant1y change. That is, it takes time to estimate the start of the motion of the user.

Thus, in a case where the motion estimation method is applied to a control device of a motion support device that supports a walking motion of a user and the like, it takes time to estimate a start of a motion of the user, it is thus difficult to quickly support the motion, and thus the motion support device is actually likely to hinder the motion of the user.

### SUMMARY

An embodiment of the disclosure is for a control device 10 of a motion support device (walking assist device 1) worn by a user M to support a motion of at least the lower body of the user M, the control device including a first motion sensor (left foot motion sensor 26) capable of detecting a motion of a left sole part of the user M, a second motion sensor (right foot motion sensor 27) capable of detecting a motion of a right sole part of the user M, a third motion sensor (waist motion sensor 28) capable of detecting a motion of a waist part of the user M, a standing state estimation part (assist controller 11, STEPS 7 and 8) estimating whether the user M is in a standing state in accordance with a detection signal of the first to third motion sensors, a waist movement state parameter calculation part (assist controller 11) calculating a waist movement state parameter (an x-axis speed of the waist part V_Wx, the absolute value of the x-axis speed of the waist part VA_Wx, or the absolute value of a y-axis speed of the waist part VA_Wy) indicating a movement state of the waist part of the user M in accordance with the detection signal of the third motion sensor, a motion estimation part (assist controller 11, STEPS 40 to 43) estimating that the user M has started a predetermined motion from the standing state if the waist movement state parameter has a value in a predetermined range indicating a movement of the waist part of the user M in one of a backward direction and a lateral direction in a case where the user M is estimated to be in the standing state, and a control part (assist controller 11, STEPS 90 to 96) controlling the motion support device such that the predetermined motion is supported in a case where the user M is estimated to have started the predetermined motion.

In an embodiment of the disclosure, if the waist movement state parameter has a value in the predetermined range indicating a movement of the waist part of the user M in the backward direction (YES in STEPS 55 and 56), the motion estimation part may estimate that the user M has started a crouching motion as the predetermined motion from the standing state (STEP 41), and the control part may control the motion support device such that the crouching motion is supported (STEPS 93 to 94) in a case where the user M is estimated to have started the crouching motion from the standing state.

In an embodiment of the disclosure, a fourth motion sensor (head motion sensor 29) capable of detecting a motion of a head part of the user M and a forward tilt angle calculation part (assist controller 11) calculating a forward tilt angle of the head part of the user M Ahead in accordance with a detection signal of the fourth motion sensor are further provided, and if the waist movement state parameter has a value in the predetermined range and the forward tilt angle of the head part of the user M Ahead has a value in a second predetermined range (YES in STEPS 51, 55 to 56) in the case where the user M is estimated to be in the standing state, the motion estimation part may estimate that the user M has started the crouching motion from the standing state (STEP 58).

In an embodiment of the disclosure, if the waist movement state parameter has a value in the predetermined range indicating a movement of the waist part in the lateral direction (YES in STEP 71) in a case where the user M is estimated to be in the standing state, the motion estimation part may estimate that the user M has started a walking motion as the predetermined motion from the standing state (STEP 73), and the control part may control the motion support device such that the walking motion is supported in a case where the user M is estimated to have started the walking motion from the standing state (STEPS 91 and 92).

In an embodiment of the disclosure, if the waist movement state parameter has a value in the predetermined range indicating the movement of the waist part in the lateral direction and a value in a third predetermined range indicating a movement of the waist part in a forward direction (YES in STEPS 70 and 71) in a case where the user M is estimated to be in the standing state, the motion estimation part may estimate that the user M has started the walking motion from the standing state.

Another embodiment of the disclosure is for a control device 10 of a motion support device (walking assist device 1) worn by a user M to support a motion of at least the lower body of the user M, the control device including a first motion sensor (left foot motion sensor 26) capable of detecting a motion of a left sole part of the user M, a second motion sensor (right foot motion sensor 27) capable of detecting a motion of a right sole part of the user M, a third motion sensor (waist motion sensor 28) capable of detecting a motion of a waist part of the user M, a fourth motion sensor (head motion sensor 29) capable of detecting a motion of a head part of the user M, a sitting state estimation part (assist controller 11, STEPS 5 and 6) estimating whether the user M is in a sitting state in accordance with a detection signal of the first to third motion sensors, a forward tilt state parameter calculation part (assist controller 11) calculating a forward tilt state parameter (forward tilt angle of the upper body θupper) indicating a forward tilt state of an upper body of the user M in accordance with the detection signal of the third motion sensor and the fourth motion sensor, a motion estimation part (assist controller 11, STEPS 85 and 87) estimating that the user M has started a standing-up motion from the sitting state if the forward tilt state parameter has a value in a fourth predetermined range indicating a forward tilt of the upper body of the user M in a case where the user M is estimated to be in the sitting state, and a control part (assist controller 11, STEPS 95 and 96) controlling the motion support device such that the standing-up motion is supported in a case where the user M is estimated to have started the standing-up motion.

According to another embodiment of the disclosure, a forward tilt angle calculation part (assist controller 11) calculating a forward tilt angle of the head part of the user M Ahead in accordance with the detection signal of the fourth motion sensor (head motion sensor 29) is further provided, and if the forward tilt state parameter has the value in the fourth predetermined range and the forward tilt angle of the head part has a value in a fifth predetermined range (YES in STEPS 81 and 85) in the case where the user M is estimated to be in the sitting state, the motion estimation part may estimate that the user M has started the standing-up motion from the sitting state (STEP 87).

### BRIEF DESCRIPTION OF THE DRAWINGS

FIG. 1 is a perspective view schematically illustrating a configuration of a control device according to an embodiment of the disclosure and a walking assist device to which the control device is applied.

FIG. 2 is a side view of the walking assist device.

FIG. 3 is a block diagram illustrating an electrical configuration of the control device.

FIG. 4 is a diagram illustrating posture change of a subject when he or she performs a crouching motion.

FIG. 5 is a diagram illustrating posture change of a subject when he or she performs a standing-up motion.

FIG. 6 is a diagram illustrating posture change of a subject when he or she performs a walking motion.

FIG. 7 is an overhead view of a side tilt posture C2 of FIG. 6.

FIG. 8 is a flowchart showing a motion state estimation process.

FIG. 9 is a flowchart showing a walking estimation process.

FIG. 10 is a flowchart showing a motion start estimation process.

FIG. 11 is a flowchart showing a crouching start estimation process.

FIG. 12 is a flowchart showing a walking start estimation process.

FIG. 13 is a flowchart showing a standing start estimation process.

FIG. 14 is a flowchart showing an assist control process.

FIG. 15 is a timing flowchart showing transitions of various parameters when a crouching motion is started.

### DESCRIPTION OF THE EMBODIMENTS

The embodiments of the disclosure provide a control device of a motion support device that can quickly and appropriately support a predetermined motion of a user when the user wearing the motion support device performs the predetermined motion.

According to the control device of the motion support device, whether the user is in a standing state is estimated in accordance with detection signals of the first to third motion sensors. In this case, since the first to third motion sensors can detect each of motions of the left sole part, the right sole part, and the waist part, whether the user is in the standing state can be accurately estimated from positional relations of the left sole part and the right sole part with the waist part. In addition, in a case where the waist movement state parameter indicating a movement state of the waist part of the user is calculated and the user is estimated to be in the standing state in accordance with a detection signal of the third motion state, if the waist movement state parameter has a value in the predetermined range indicating a movement of the waist part of the user in one of the backward direction and the lateral direction, the user is estimated to have started the predetermined motion from the standing state.

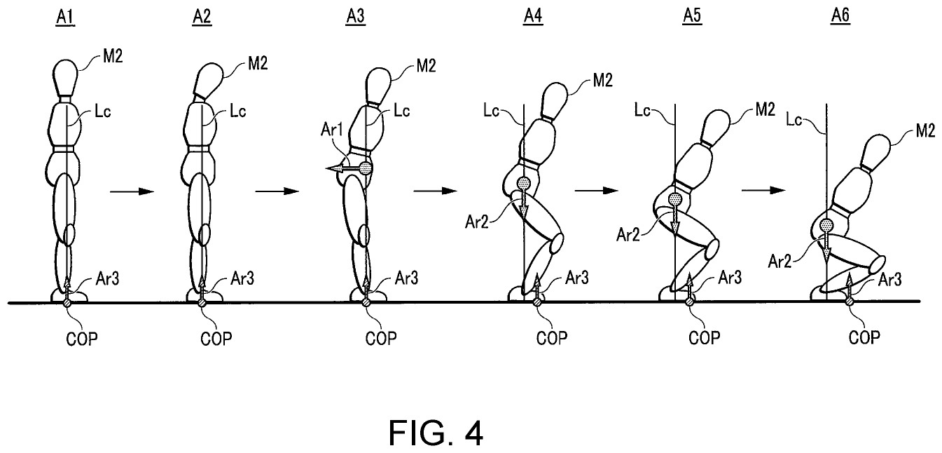

In this case, the present applicants have ascertained through testing that, when a human starts a predetermined motion (e.g., a crouching motion or a walking motion) from a standing state, a motion of the waist part moving in the backward direction or the lateral direction is performed first (see FIGS. 4, 6, and 7 which will be described below). Therefore, whether the user has started a predetermined motion from a standing state can be accurately estimated under the condition that the waist movement state parameter has a value in the predetermined range indicating a movement of the waist part of the user in one of the backward direction or the lateral direction. Furthermore, since the motion support device is controlled such that the predetermined motion is supported in the case where the user is accurately estimated to have started the predetermined motion from the standing state as described above, the motion support device can quickly and appropriately support the predetermined motion of the user.

The present applicants have ascertained through testing that, when the user starts the crouching motion from the standing state, a motion of the waist part moving in the backward direction is performed first as described above (see FIG. 4 which will be described below). Therefore, according to the control device of the motion support device, since the user is estimated to have started the crouching motion as the predetermined motion from the standing state if the waist movement state parameter has a value in the predetermined range indicating a movement of the waist part of the user in the backward direction, the estimation can be accurately performed. Furthermore, since the motion support device is controlled such that the crouching motion is supported in the case where the user is accurately estimated to have started the crouching motion as described above, the motion support device can quickly and appropriately support the crouching motion when the user starts the crouching motion (further, “crouching motion” in the present specification means a series of motions of the user performed from a standing state to be in a completely crouching state).

According to the control device of the motion support device, the user is estimated to have started the crouching motion from the standing-up motion if the waist movement state parameter has a value in the predetermined range and the forward tilt angle of the head part of the user has a value in the second predetermined range in the case where the user is estimated to be in the standing state. In this case, the present applicants have ascertained through testing that, when a human starts a crouching motion from a standing state, a forward tilt motion of the head part of the user accompanies a motion of the waist part of the user moving in the backward direction as described above (see FIG. 4 which will be described below). Therefore, since a start of the crouching motion from the standing state is estimated under the condition of a forward tilt motion of the head part in addition to a motion of the waist part of the user moving in the backward direction as described above, the estimation accuracy can be further improved. Thus, control accuracy of the motion support device can be further improved.

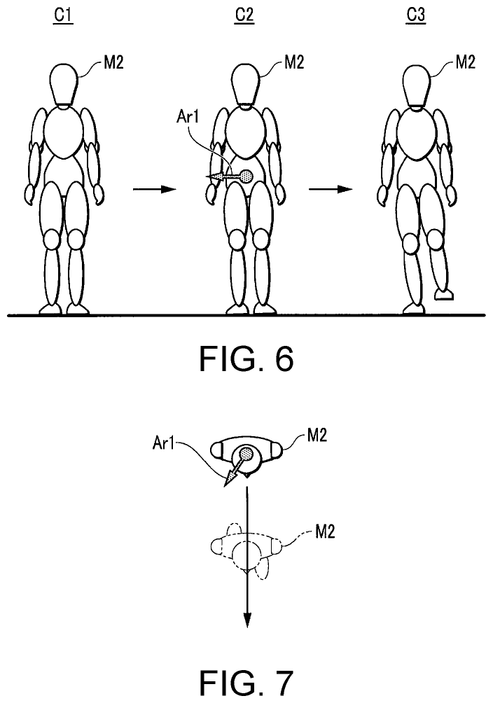

The present applicants have ascertained through testing that, when a human starts a walking motion from a standing state, a motion of the waist moving in the lateral direction is performed first as described above (see FIGS. 6 and 7 which will be described below). Therefore, according to the control device of the motion support device, since the user is estimated to have started the walking motion as a predetermined motion from the standing state if the waist movement state parameter has a value in the predetermined range indicating a movement of the waist part in the lateral direction, the estimation can be accurately performed. Furthermore, since the motion support device is controlled such that the walking motion is supported in a case where the user is accurately estimated to have started the walking motion as described above, the motion support device can quickly and appropriately support the walking motion when the user starts the walking motion (further, “walking motion” in the present specification means a series of motions of the user performed from a standing state to be in a completely walking state).

The present applicants have ascertained through testing that, when a human starts a walking motion from a standing state, a movement of the waist part in the forward direction is performed first in addition to a movement of the waist part in the lateral direction as described above (see FIG. 7 which will be described below). Therefore, according to the control device of the motion support device, since the user is estimated to have started the walking motion from the standing state if the waist movement state parameter has the value in the predetermined range indicating the movement of the waist part in the lateral direction and the value in the third predetermined range indicating the movement of the waist part in the forward direction, the estimation accuracy can be further improved. Accordingly, control accuracy of the motion support device can be further improved.

According to the control device of the motion support device, whether the user is in a sitting state is estimated in accordance with detection signals of the first to third motion sensors. In this case, since the first to third motion sensors can detect motions of the left sole part, the right sole part, and the waist part, the user can be accurately estimated to be in the sitting state from positional relations of the left sole part and the right sole part with the waist part. In addition, in a case where the forward tilt state parameter indicating a forward tilt state of the upper body of the user is calculated and the user is estimated to be in the sitting state in accordance with detection signals of the third and fourth motion state, if the forward tilt state parameter has a value in the fourth predetermined range indicating a movement of the upper body of the user tilting forward, the user is estimated to have started the standing-up motion as the predetermined motion from the sitting state.

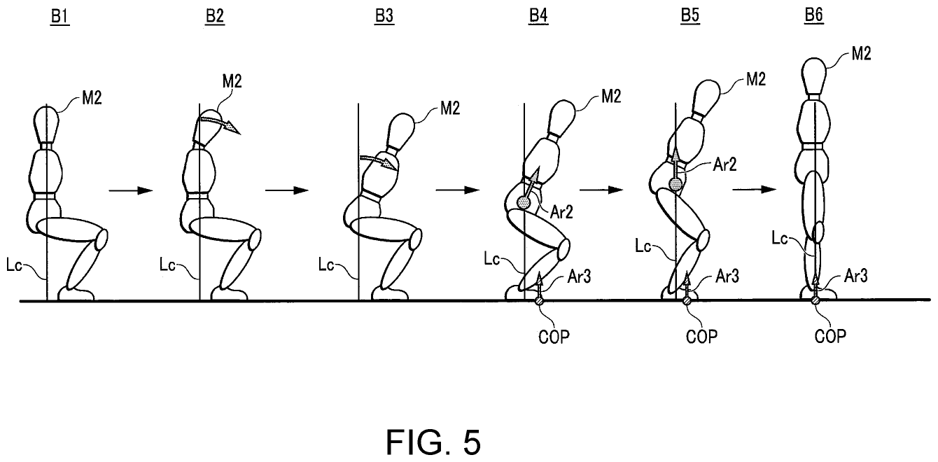

In this case, the present applicants have ascertained through testing that, when a human starts a standing-up motion from a sitting state, a motion of tilting the upper body forward, that is, a motion of moving the head part and the waist part away from each other in the front-rear direction, is performed first (see FIG. 5 which will be described below). Therefore, whether the user has started the standing-up motion from the sitting state can be accurately estimated under the condition that the forward tilt state parameter has a value in the fourth predetermined range indicating that the user is tilting his or her upper body forward. Furthermore, since the motion support device is controlled such that the standing-up motion is supported in a case where the user is accurately estimated to have started the standing-up motion, the motion support device can quickly and appropriately support the standing-up motion when the user starts the standing-up motion (further, “standing-up motion” in the present specification means a series of motions of the user performed from a sitting state to be in a completely standing state).

According to the control device of the motion support device, if the forward tilt state parameter has the value in the fourth predetermined range and the forward tilt angle of the head part has the value in the fifth predetermined range in the case where the user M is estimated to be in the sitting state, the user is estimated to have started the standing-up motion from the sitting state. In this case, the present applicants have ascertained through testing that, when a human starts a standing-up motion from a sitting state, a forward tilt motion of the head part of the user accompanies a forward tilt motion of the upper body of the user (see FIG. 5 which will be described below). Therefore, since a start of the standing-up motion from the sitting state is estimated under the condition of the forward tilt motion of the head part in addition to the forward tilt motion of the upper body of the user, the estimation accuracy can be further improved. Therefore, control accuracy of the motion support device can be further improved.

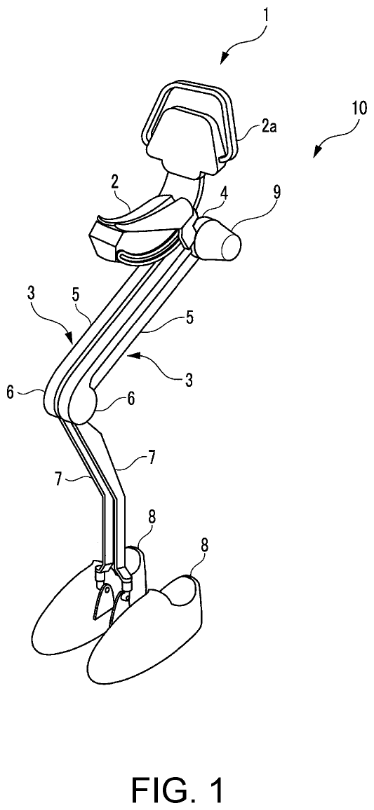

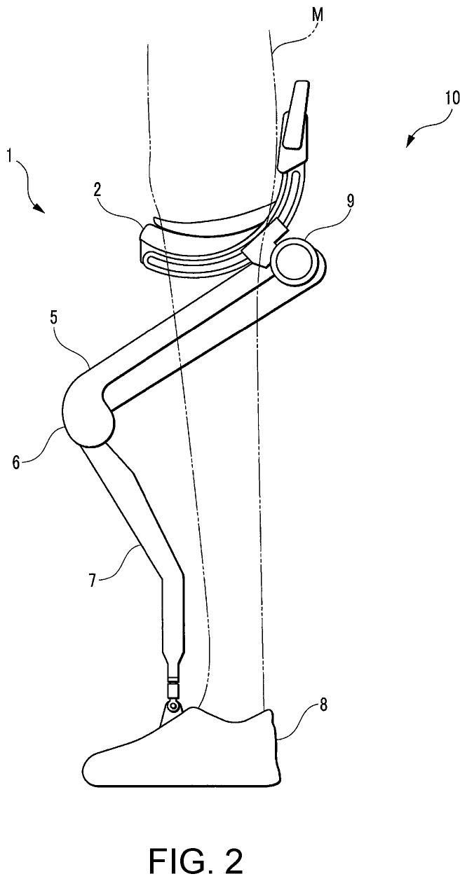

A control device of a motion support device according to an embodiment of the disclosure will be described below with reference to the drawings. The control device 10 of the present embodiment controls states of motions of a walking assist device 1 serving as a motion support device as illustrated in FIG. 1 and FIG. 2, and first, the walking assist device 1 will be described below.

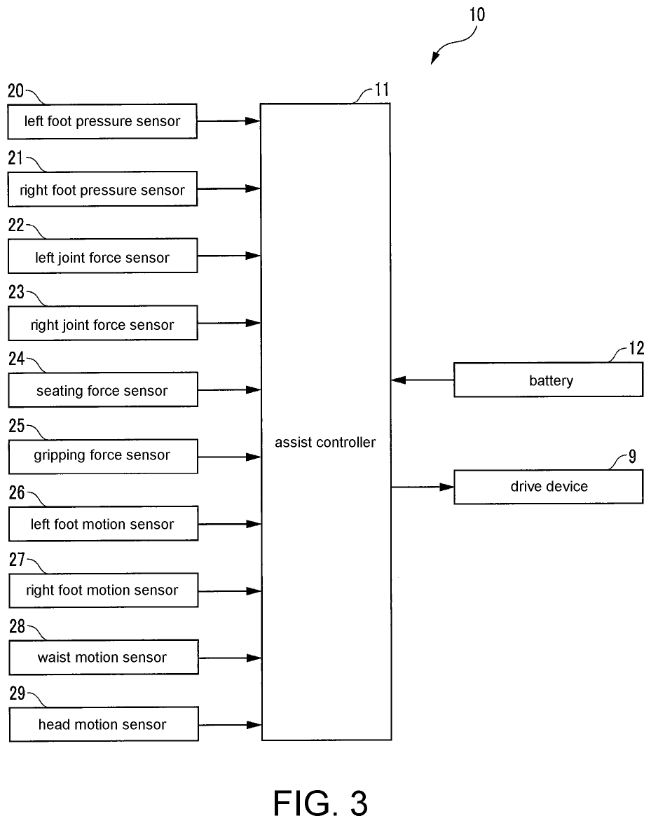

The walking assist device 1 assists a user M with walking motions and the like and is of an active type including a drive device 9 (see FIG. 3) as a power source. Further, in the following description, a front-rear direction of the walking assist device 1 and the user M who is wearing the walking assist device will be referred to as “front-rear,” a left-right direction thereof will be referred to as “left-right,” and a top-bottom direction thereof will be referred to as “top-bottom.”

Although the walking assist device 1 is configured similarly to, specifically, that disclosed in Japanese Patent No. 4872821, for example, and detailed description thereof will be omitted here, a seat member 2 and a pair of left and right leg link mechanisms 3 and 3 are provided. The user M is seated on the seat member 2 while wearing the walking assist device 1.

In addition, each of the leg link mechanisms 3 and 3 includes a first joint 4, a first link member 5, a second joint 6, and a second link member 7. The first link member 5 is connected to the seat member 2 to be capable of freely swinging via the first joint 4. Furthermore, the first link member 5 is connected to the second link member 7 to be capable of freely rotating via the second joint 6.

In addition, a shoe-shaped grounding member 8 is connected at a lower end of the second link member 7 of each leg link mechanism 3. When the user M wears the walking assist device 1, the left and right sole parts of the user M are inserted into the grounding member 8.

Furthermore, a drive device 9 is attached to the leg link mechanism 3. The drive device 9 is a combination of a motor and a reduction gear mechanism (neither of which is illustrated) and is electrically connected to an assist controller 11. The drive device 9 drives an angle between the second link member 7 and the first link member 5 to change by being controlled by the assist controller 11 as will be described below. Accordingly, an assisting force for supporting a body weight of the user M is generated and thus the user M can be assisted with walking.

Next, the control device 10 will be described with reference to FIG. 3. As illustrated in the drawing, the control device 10 includes the assist controller 11 and a battery 12, and both the assist controller 11 and the battery 12 are built in the seat member 2.

The assist controller 11 is configured as a microcomputer including a CPU, a RAM, a ROM, an I/O interface, a wireless communication circuit, various electric circuits (none of which are illustrated), and the like, and operates by receiving supply of power from the battery 12. The ROM stores various programs for executing a motion state estimation process, and the like, which will be described below.

Further, in the present embodiment, the assist controller 11 corresponds to a standing state estimation part, a waist movement state parameter calculation part, a motion estimation part, a control part, a forward tilt angle calculation part, a sitting state estimation part, and a forward tilt state parameter calculation part.

The assist controller 11 is electrically connected to a left foot pressure sensor 20, a right foot pressure sensor 21, a left joint force sensor 22, a right joint force sensor 23, a seating force sensor 24, a gripping force sensor 25, a left foot motion sensor 26, a right foot motion sensor 27, a waist motion sensor 28, and a head motion sensor 29.

The left foot pressure sensor 20 and the right foot pressure sensor 21 are built in the bottoms of the left and right grounding members 8 and 8, respectively, detect pressure acting on the bottoms of the left and right grounding members 8 and 8, and output detection signals indicating the pressure to the assist controller 11. The assist controller 11 determines the left and right sole parts of the user M to be in contact with the grounding member 8 on the basis of the detection signals of the left and right foot pressure sensors 20 and 21.

In addition, the left joint force sensor 22 and right joint force sensor 23 are provided in the left and right second joints 6 and 6, respectively, detect forces acting on the joints, and output detection signals indicating the forces to the assist controller 11.

Furthermore, the seating force sensor 24 detects a force acting between the seat member 2 and the thighs of the user M and outputs a detection signal indicating the force to the assist controller 11, and the gripping force sensor 25 detects a force acting on a grip part 2a of the seat member 2 and outputs a detection signal indicating the force to the assist controller 11.

Meanwhile, the left foot motion sensor 26 and the right foot motion sensor 27 are of an inertial measurement unit type, are provided on the sole parts of the left and right grounding members 8 and 8, and are configured to be capable of performing wireless communication with the assist controller 11. The left and right foot motion sensors 26 and 27 detect three-axis (x, y, and z axes) accelerations, three-axis rotation angles, and three-axis positions of the left and right grounding members 8 and 8 and output detection signals indicating the values to the assist controller 11 as radio signals.

The assist controller 11 computes three-axis speeds, positions, and the like of the left and right sole parts of the user M on the basis of the detection signals from the left and right foot motion sensors 26 and 27. Further, in the present embodiment, the left foot motion sensor 26 corresponds to a first motion sensor, and the right foot motion sensor 27 corresponds to a second motion sensor.

In addition, the waist motion sensor 28 is of an inertial measurement unit type as well, and is configured to be worn around the waist part of the user M in the form of a belt or the like and capable of wirelessly communicating with the assist controller 11. The waist motion sensor 28 detects a three-axis (x, y, and z axes) acceleration, a three-axis rotation angle, and a three-axis position of the waist part of the user M and outputs detection signals indicating the values to the assist controller 11 as radio signals.

The assist controller 11 calculates a three-axis speed, position, and the like of the waist part of the user M on the basis of the detection signals of the waist motion sensor 28. Further, in the present embodiment, the waist motion sensor 28 corresponds to a third motion sensor.

Furthermore, the head motion sensor 29 is of an inertial measurement unit type as well, and is configured to be worn on the top of the head of the user M in the form of a hat or the like and capable of wirelessly communicating with the assist controller 11. The head motion sensor 29 detects a three-axis (x, y, and z axes) acceleration, a three-axis rotation angle, and a three-axis position of the top of the head of the user M and outputs detection signals indicating the values to the assist controller 11 as radio signals.

The assist controller 11 calculates a tilt angle, a position, and the like of the head part of the user M on the basis of the detection signals of the head motion sensor 29. In this case, the tilt angle of the head part of the user M is calculated to indicate a positive value in a forward tilting direction, that is, a bowing direction. Further, in the present embodiment, the head motion sensor 29 corresponds to a fourth motion sensor.

In addition, with respect to the four above-described motion sensors 26 to 29, when the user M wearing the walking assist device 1 is in a standing state and the attachment positions of the sensors are set to the origin, the front-rear direction in a room coordinate system is set as an x-axis direction, the left-right direction is set as a y-axis direction, and the top-bottom direction is set as a z-axis direction. In addition, with respect to each of the sensors, a detection value forward from the origin in the x-axis direction is set as a positive value, a detection value rearward from the origin in the x-axis direction is set as a negative value, and a detection value to the left of the origin in the y-axis direction is set as a positive value and a detection value to the right of the origin in the y-axis direction is set as a negative value. Furthermore, a detection value above the origin in the z-axis direction is set as a positive value, and a detection value below the origin in the z-axis direction is set as a negative value.

The assist controller 11 causes a motion estimation process to be performed in accordance with detection signals of the four above-described motion sensors 26 to 29, and as will be described below, causes an assist control process to be performed in accordance with detection signals of the ten sensors 20 to 29.

Next, the principle of motion estimation by the assist controller 11 will be described. First, the principle of the method for estimating whether the user M has started a crouching motion from a standing state will be described with reference to FIG. 4. The drawing illustrates change in posture of a healthy subject M2 who does not need the walking assist device 1 acquired using a motion capture method in the case where the subject repeats a crouching motion from a standing state many times to be in a crouching state and an average of the change.

In the drawing, COP represents an application point of a floor reaction force, and Lc represents a vertical line (i.e., a z-axis line) passing the origin of the x, y, and z axes of the waist motion sensor 28. In addition, the arrow Art represents a movement speed of the waist part in the x-axis direction, the arrow Ar2 represents a movement speed of the waist part in the z-axis direction, and the arrow Ar3 extending upward from the application point of a floor reaction force COP represents a reaction force from the floor. The above-described matters also apply to FIGS. 5 to 7, which will be described below.

In a case where the subject M2 performs a crouching motion from a standing state as illustrated in FIG. 4, the subject M2 first tilts only his or her head part from the standing posture A1, and thereby the posture changes to a looking-down posture A2. Next, when the subject M2 tilts his or her upper body forward while moving his or her waist backward from the looking-down posture A2, the posture transitions to the forward tilt posture A3. Then, when the subject M2 drops his or her waist from the forward tilt posture A3, the posture transitions to a middle waist posture A4, and when the subject further drops his or her waist, the posture transitions to a middle waist posture A5.

In addition, when the subject M2 further drops his or her waist from the middle waist posture A5, the posture of the subject finally reaches a crouching posture A6. Since the crouching motion is performed as described above, it is ascertained that, in a case where whether the user M has started a crouching motion from a standing state is to be estimated, it is good to determine whether a posture of the user M has transitioned from the standing posture A1 to the forward tilt posture A3. Based on the above-described principle, a start of a crouching motion is estimated in the present embodiment using an estimation method which will be described below.

Next, the principle of a method for estimating whether the user M has started a standing-up motion from a sitting state will be described with reference to FIG. 5. The drawing illustrates change in posture of the above-described subject M2 acquired using a motion capture method in the case where the subject repeats a standing-up motion performed from a sitting state many times to be in a standing state and an average of the change.

As illustrated in the drawing, in a case where the subject M2 performs a standing-up motion from a sitting state, the subject M2 first tilts only his or her head part forward from a sitting posture B1, and thereby the posture changes to a looking-down posture B2. Next, when the subject M2 tilts his or her upper body forward from the looking-down posture B2, the posture transitions to a forward tilt posture B3. Then, when the subject M2 moves his or her waist obliquely upward from the forward tilt posture B3, the posture of the subject M2 transitions to a middle waist posture B4, and when the subject moves his or her waist upward from the middle waist posture B4, the posture transitions to a middle waist posture B5.

Then, when the subject M2 moves his or her waist further upward from the middle waist posture B5, the posture of the subject M2 finally reaches a standing posture B6. Since the standing-up motion is performed as described above, it is ascertained that, in a case where whether the user M has started a standing-up motion from a sitting state is to be estimated, it is good to determine whether a posture of the user M has transitioned from the sitting posture B1 to the forward tilt posture B3. Based on the above-described principle, a start of a standing-up motion can be estimated in the present embodiment using the estimation method which will be described below.

Next, the principle of a method for estimating whether the user M has started a walking motion from a standing state will be described with reference to FIG. 6 and FIG. 7. FIG. 6 illustrates change in posture of the above-described subject M2 acquired using a motion capture method when the subject repeats a walking start motion many times from a standing state to be in a walking state and an average of the change, and FIG. 7 illustrates a side tilt posture C2 of FIG. 6 viewed from above.

In a case where the subject M2 performs a walking motion from a standing state as illustrated in FIG. 6, when the subject M2 first moves his or her waist part obliquely forward from a standing posture C1 (forward and obliquely to the right in FIG. 6 and FIG. 7), the posture changes to the side tilt posture C2. Next, when the subject M2 raises his or her foot on the side opposite to the direction in which the waist was moved, the posture transitions to a foot-up posture C3. Then, although not illustrated, when the subject M2 moves his or her waist toward his or her foot while putting the raised foot on the floor, the subject M2 transitions to a walking motion state.

Since the subject M2 performs a walking motion as described above, it is ascertained that it is good to determine whether a posture of the user M has transitioned from the standing posture C1 to the side tilt posture C2. Based on the above-described principle, a start of a walking motion is estimated in the present embodiment using the estimation method which will be described below.

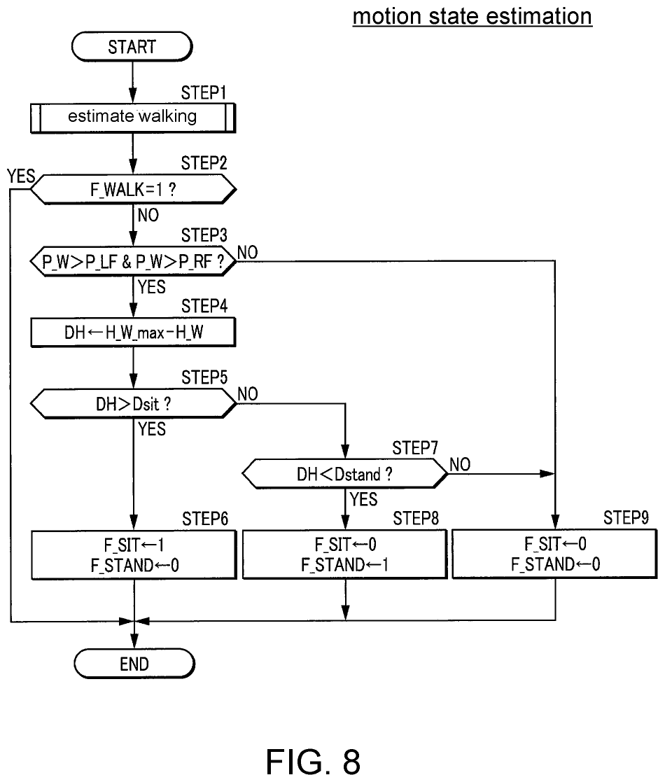

Next, the motion state estimation process will be described with reference to FIG. 8. The motion state estimation process is to estimate a motion state of a user M wearing the walking assist device 1 (which will be referred to simply as a “user M” below) and is performed by the assist controller 11 on a predetermined control cycle. Further, various values calculated and set in the following description are assumed to be stored in the RAM of the assist controller 11.

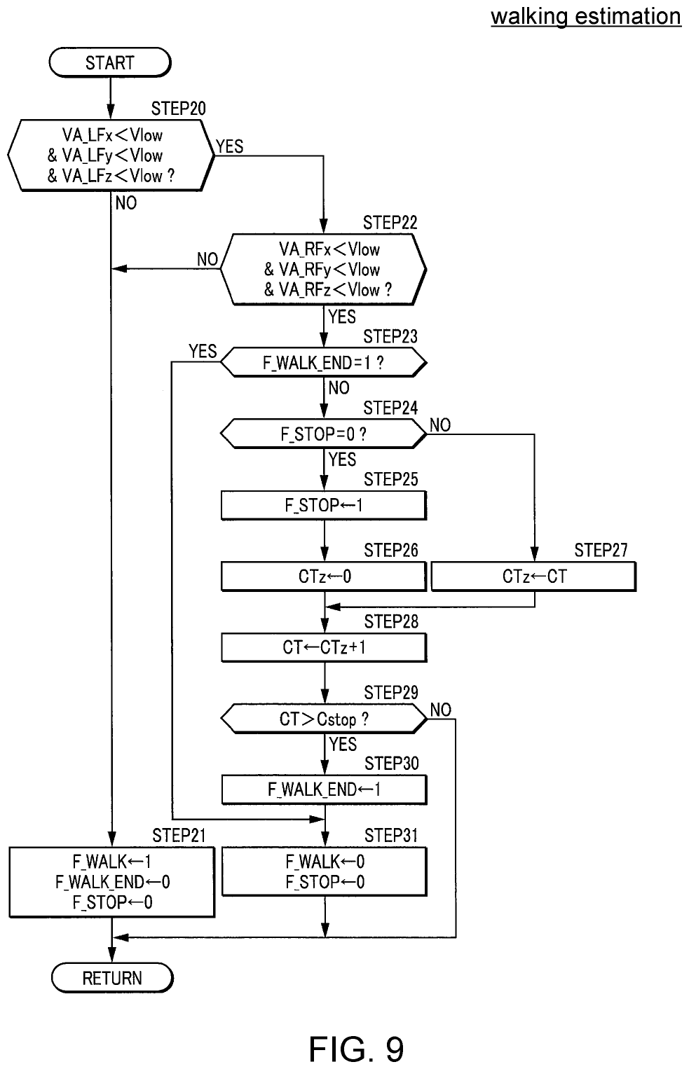

First, a walking estimation process is performed as shown in the drawing (FIG. 8/STEP 1). The walking estimation process is to estimate whether the user M is in a walking state, and specifically, performed as illustrated in FIG. 9.

First, whether all of VA_LFx<Vlow, VA_LFy<Vlow, and VA_LFz<Vlow are satisfied is determined as shown in the drawing (FIG. 9/STEP 20). In this case, VA_LFx represents the absolute value of an x-axis speed of the left sole part of the user M, VA_LFy represents the absolute value of a y-axis speed of the left sole part of the user M, and VA_LFz represents the absolute value of a z-axis speed of the left sole part of the user M, and these values are calculated on the basis of detection signals of the left foot motion sensor 26. In addition, Vlow represents a positive predetermined value satisfying Vlow≈0.

If the result of the determination is negative (FIG. 9/NO in STEP 20), that is, if the left sole part of the user M is in a moving state, the user M is estimated to be walking, and a walking flag F_WALK is set to “1,” and at the same time both a walking end flag F_WALK_END and a stop flag F_STOP are set to “0” to indicate the state (FIG. 9/STEP 21). Then, the process ends.

On the other hand, if the result of the determination is positive (FIG. 9/YES in STEP 20), that is, if the left sole part of the user M is in a stop state, it is determined whether all of VA_RFx<Vlow, VA_RFy<Vlow, and VA_RFz<Vlow are satisfied (FIG. 9/STEP 22). In this case, VA_RFx represents the absolute value of an x-axis speed of the right sole part of the user M, VA_RFy represents the absolute value of a y-axis speed of the right sole part of the user M, and VA_RFz represents the absolute value of a z-axis speed of the right sole part of the user M, and these values are calculated on the basis of detection signals of the right foot motion sensor 27.

If the result of the determination is negative (FIG. 9/NO in STEP 22), that is, if the right sole part of the user M is in a moving state, the user M is estimated to be walking, and to indicate the state, the walking flag F_WALK is set to “1,” and at the same time, both the walking end flag F_WALK_END and the stop flag F_STOP are set to “0” as described above (FIG. 9/STEP 21). Then, the present process ends.

On the other hand, if the result of the above-described determination is positive (FIG. 9/YES in STEP 22), that is, if both the left sole part and the right sole part of the user M are in the stop state, whether the walking end flag F_WALK_END is “1” is determined (FIG. 9/STEP 23).

If the result of the determination is negative (FIG. 9/NO in STEP 23), whether the stop flag F_STOP stored in the RAM is “0” is determined (FIG. 9/STEP 24).

If the result of the determination is positive (FIG. 9/YES in STEP 24), that is, if both the left sole part and the right sole part of the user M are in the stop state at the current control timing, the stop flag F_STOP is set to “1” to indicate the state (FIG. 9/STEP 25). Next, the previous counted value CTz of the stop counter is set to “0” (FIG. 9/STEP 26).

On the other hand, if the result of the determination is negative (FIG. 9/NO in STEP 24), that is, if both the left sole part and the right sole part of the user M had been in the stop state at the control timing before the previous timing, the previous counted value CTz of the stop counter is set to a current counted value CT of the stop counter stored in the RAM (FIG. 9/STEP 27).

As described above, after the previous counted value CTz of the stop counter is set to the value “0” or the current value “CT,” the current counted value CT of the stop counter is set to the sum of the previous value CTz and the value “1” CTz+1 (FIG. 9/STEP 28). That is, the current counted value CT of the stop counter is incremented by “1.”

Next, whether the current counted value CT of the stop counter is greater than a predetermined stop value Cstop is determined (FIG. 9/STEP 29). If the result of the determination is negative (FIG. 9/NO in STEP 29), the present process ends as it is.

On the other hand, if the result of the determination is positive (FIG. 9/YES in STEP 29), the user M is determined to have ended the walking, and the walking end flag F_WALK_END is set to “1” to indicate the state (FIG. 9/STEP 30).

If the walking end flag F_WALK_END is set to “1” as described above or the result of the above-described determination is positive (FIG. 9/YES in STEP 23), successively, the walking flag F_WALK is set to “0” and at the same time the stop flag F_STOP is reset to “0” to indicate that the user M is not walking (FIG. 9/STEP 31). Then, the present process ends.

Returning to FIG. 8, after the walking estimation process (FIG. 8/STEP 1) is performed as described above, whether the above-described walking flag F_WALK is “1” is determined (FIG. 8/STEP 2). If the result of the determination is positive (FIG. 8/YES in STEP 2), that is, if the user M is estimated to be walking, the present process ends as it is.

On the other hand, if the result of the determination is negative (FIG. 8/NO in STEP 2) and the user M is estimated not to be walking, whether both P_W>P_LF and P_W>P_RF are satisfied is determined (FIG. 8/STEP 3). In this case, P_W represents a position of the waist part of the user M and is calculated on the basis of a detection signal of the waist motion sensor 28. In addition, P_LF represents a position of the left sole part of the user M and is calculated on the basis of a detection signal of the left foot motion sensor 26. Furthermore, P_RF represents a position of the right sole part of the user M and is calculated on the basis of a detection signal of the right foot motion sensor 27.

If the result of the determination is positive (FIG. 8/YES in STEP 3), a waist part height deviation DH is set to a deviation H_W max-H_W of a maximum waist part height H_W_max and a waist part height H_W (FIG. 8/STEP 4). The maximum waist part height H_W_max represents a height of the waist part of the user M when the user M is in a standing state and is set at the time of initialization process when the user M wears the walking assist device 1. In addition, the waist part height H_W is a current height of the waist part of the user M and is calculated on the basis of a detection signal of the waist motion sensor 28.

Next, whether the waist part height deviation DH is greater than a predetermined sitting determination value Dsit is determined (FIG. 8/STEP 5). If the result of the determination is positive (FIG. 8/YES in STEP 5), the user M is estimated to be in a sitting state and a sitting state flag F_SIT is set to “1” and a standing state flag F_STAND is set to “0” to indicate the state (FIG. 8/STEP 6). Then, the present process ends.

On the other hand, if the result of the determination is negative (FIG. 8/NO in STEP 5) and DH<Dsit is satisfied, whether the waist part height deviation DH is greater than a predetermined standing determination value Dstand is determined (FIG. 8/STEP 7). The standing determination value Dstand is set to satisfy Dstand<Dsit.

If the result of the determination is positive (FIG. 8/YES in STEP 7), the user M is estimated to be in a standing state, and the standing state flag F_STAND is set to “1” and the sitting state flag F_SIT is set to “0,” respectively, to indicate the state (FIG. 8/STEP 8). Then, the present process ends.

On the other hand, if the result of the above-described determination is negative and at least one of P_W≤P_LF and P_W≤P_RF is satisfied (FIG. 8/NO in STEP 3) or Dsit≤DH<Dstand is satisfied (FIG. 8/NO in STEP 7), the user M is estimated to be in neither a sitting state nor a standing state, and both the sitting state flag F_SIT and the standing state flag F_STAND are set to “0” (FIG. 8/STEP 9) to indicate the state. Then, the present process ends.

As described above, if the user M is estimated to be in a walking state in the motion state estimation process, the walking flag F_WALK is set to “1,” if the user M is estimated to be in a sitting state, the sitting state flag F_SIT is set to “1,” and if the user M is estimated to be in a standing state, the standing state flag F_STAND is set to “1.”

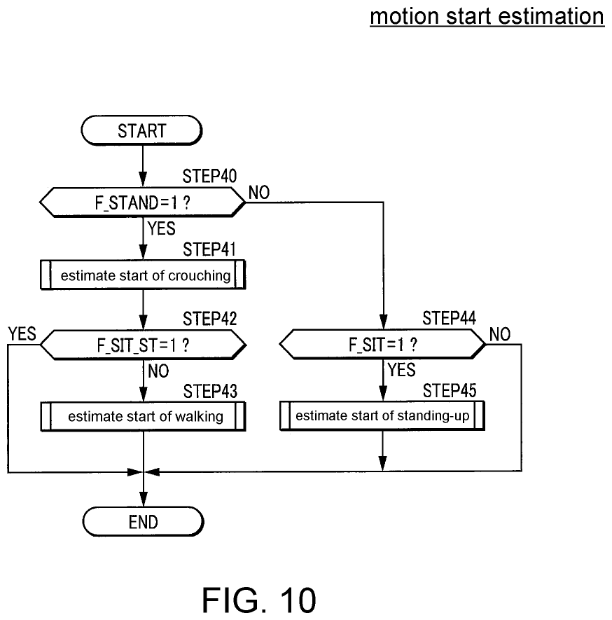

Next, a motion start estimation process will be described with reference to FIG. 10. The motion start estimation process is to estimate a start of a motion of a user M wearing the walking assist device 1 (which will be referred to simply as a “user M” below) using a method based on the above-described estimation principle and is performed by the assist controller 11 on a predetermined control cycle.

First, whether the above-described standing state flag F_STAND is “1” is determined as shown in the drawing (FIG. 10/STEP 40). If the result of the determination is positive (FIG. 8/YES in STEP 40), that is, if the user M is estimated to be in a standing state, a crouching start estimation process is performed (FIG. 10/STEP 41).

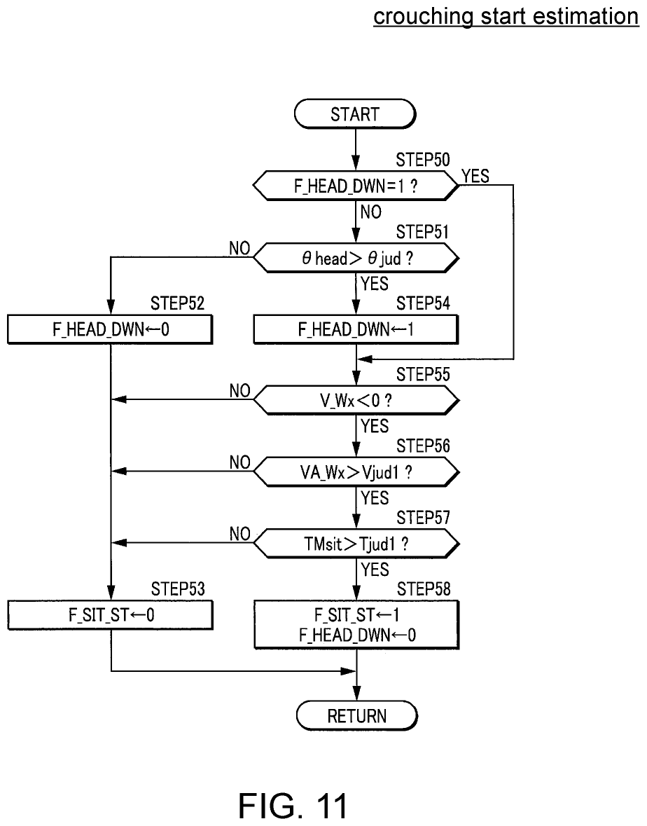

The crouching start estimation process is to estimate whether the user M, who is in a standing state, has started a crouching motion and is performed specifically as shown in FIG. 11. First, whether a head part forward tilt flag F_HEAD_DWN is “1” is determined as shown in the drawing (FIG. 11/STEP 50).

If the result of the determination is negative (FIG. 11/NO in STEP 50), whether θhead>θjud is satisfied is determined (FIG. 11/STEP 51). Ahead represents a forward tilt angle of the head part of the user M and is calculated on the basis of a detection signal of the head motion sensor 29. In addition, θjud is a determination value for determining whether the user M looks down in a forward direction.

If the result of the determination is negative (FIG. 11/NO in STEP 51) and the user M is not looking down in the forward direction, the head part forward tilt flag F_HEAD_DWN is set to “0” to indicate the state (FIG. 11/STEP 52). Next, the user M is estimated not to be starting a crouching motion and a crouching start flag F_SIT_ST is set to “0” to indicate the state (FIG. 11/STEP 53). Then, the present process ends.

On the other hand, if the result of the determination is positive (FIG. 11/YES in STEP 51) and θhead>θjud is satisfied, the user M is estimated to be looking down in the forward direction and the head part forward tilt flag F_HEAD_DWN is set to “1” to indicate the state (FIG. 11/STEP 54).

If the head part forward tilt flag F_HEAD_DWN is set to “1” or if the result of the above-described determination is positive (FIG. 11/YES in STEP 50) and the head part forward tilt flag F_HEAD_DWN is set to “1” at the timing prior to the previous timing as described above, whether V_Wx<0 is satisfied is successively determined (FIG. 11/STEP 55). V_Wx is an x-axis speed of the waist part of the user M and is calculated on the basis of a detection signal of the waist motion sensor 28.

If the result of the determination is negative (FIG. 11/NO in STEP 55), the crouching start flag F_SIT_ST is set to “0” as described above (FIG. 11/STEP 53), and then the present process ends.

On the other hand, if the result of the determination is positive (FIG. 11/YES in STEP 55), that is, the user M is estimated to be moving his or her waist part backward, whether VA_Wx>Vjud1 is satisfied is determined (FIG. 11/STEP 56). VA_Wx is the absolute value of the x-axis speed of the waist part of the user M, and Vjud1 represents a predetermined determination value for determining whether the user M is actually moving his or her waist part backward. Further in the present embodiment, the x-axis speed V_Wx and the absolute value VA_Wx of the waist part correspond to waist movement state parameters.

If the result of the determination is negative (FIG. 11/NO in STEP 56), the crouching start flag F_SIT ST is set to “0” as described above (FIG. 11/STEP 53), and then the present process ends.

On the other hand, if the result of the determination is positive (FIG. 11/YES in STEP 56), that is, if the user M is estimated to be actually moving his or her waist part backward, whether TMsit>Tjud1 is satisfied is determined (FIG. 11/STEP 57). TMsit represents a time elapsed in a state in which V_Wx<0 and VA_Wx>Vjud1 are satisfied, and Tjud1 represents a predetermined determination value for determining whether the user M is actually moving his or her waist part backward.

If the result of the determination is negative (FIG. 11/NO in STEP 57), the crouching start flag F_SIT_ST is set to “0” as described above (FIG. 11/STEP 53), and then the present process ends.

On the other hand, if the result of the determination is positive (FIG. 11/YES in STEP 57), the user M is estimated to have started a crouching motion, and the crouching start flag F_SIT_ST is set to “1,” and at the same time, the head part forward tilt flag F_HEAD_DWN is reset to “0” to indicate the state (FIG. 11/STEP 58). Then, the present process ends.

Returning to FIG. 10, after the crouching start estimation process (FIG. 10/STEP 41) is performed as described above, whether the above-described crouching start flag F_SIT_ST is “1” is determined (FIG. 10/STEP 42). If the result of the determination is positive (FIG. 10/YES in STEP 42) and the user M is estimated to have started the crouching motion, the present process ends as it is.

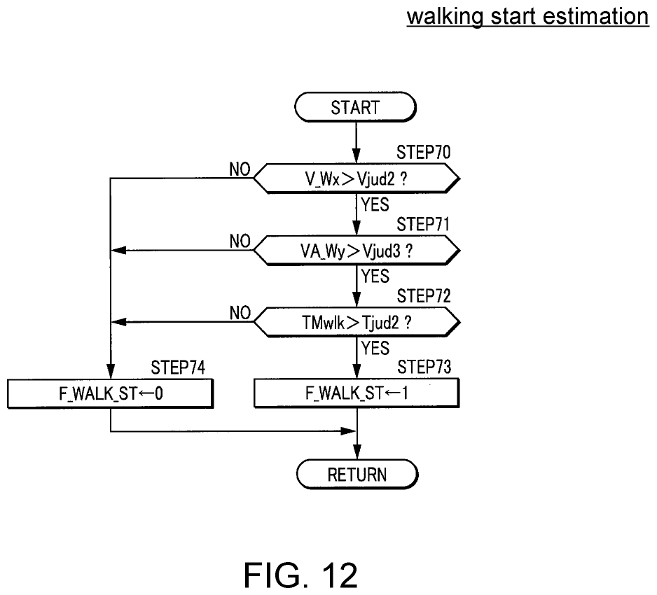

On the other hand, if the result of the determination is negative (FIG. 10/NO in STEP 42), a walking start estimation process is performed (FIG. 10/STEP 43). This walking start estimation process is to estimate whether the user M who is in a standing state has started a walking motion and is performed specifically as shown in FIG. 12.

First, whether V_Wx>Vjud2 is satisfied is determined as shown in the drawing (FIG. 12/STEP 70). Vjud2 is a determination value for determining whether the waist part of the user M is actually moving forward.

If the result of the determination is negative (FIG. 12/NO in STEP 70), that is, if the waist part of the user M is estimated not to be moving forward, the user M is estimated not to have started a walking motion, and a walking start flag F_WALK_ST is set to “0” to indicate the state (FIG. 12/STEP 74). Then, the present process ends.

On the other hand, if the result of the determination is positive (FIG. 12/YES in STEP 70), whether VA_Wy>Vjud3 is satisfied is determined (FIG. 12/STEP 71). VA_Wy represents the absolute value of a y-axis speed of the waist part of the user M, and Vjud3 represents a predetermined determination value for determining whether the user M is actually moving his or her waist part in the left-right direction. Further, in the present embodiment, the absolute value of the y-axis speed of the waist part VA_Wy corresponds to a waist movement state parameter.

If the result of the determination is negative (FIG. 12/NO in STEP 71), the walking start flag F_WALK_ST is set to “0” (FIG. 12/STEP 74) as described above, and then the present process ends.

On the other hand, if the result of the determination is positive (FIG. 12/YES in STEP 71), and if the user M is estimated to be actually moving his or her waist part in the left-right direction, whether TMwlk>Tjud2 is satisfied is determined (FIG. 12/STEP 72). TMwlk represents a time elapsed in a state in which V_Wx>Vjud2 and VA_Wy>Vjud3 are satisfied, and Tjud2 represents a predetermined determination value for determining whether the user M is actually moving his or her waist part obliquely forward.

If the result of the determination is negative (FIG. 11/NO in STEP 72), the walking start flag F_WALK_ST is set to “0” (FIG. 12/STEP 74) as described above, and then the present process ends.

On the other hand, if the result of the determination is positive (FIG. 11/YES in STEP 72), the user M is estimated to have started a walking motion, and the walking start flag F_WALK_ST is set to “1” to indicate the state (FIG. 12/STEP 73). Then, the present process ends.

Returning to FIG. 10, after the walking start estimation process (FIG. 10/STEP 43) is performed as described above, the motion start estimation process ends.

On the other hand, if the result of the above-described determination is negative (FIG. 10/NO in STEP 40), that is, the standing state flag F_STAND is “0,” whether the above-described sitting state flag F_SIT is “1” is determined (FIG. 10/STEP 44). If the result of the determination is negative (FIG. 10/NO in STEP 44), that is, the user M is neither in a standing state nor a sitting state, the present process ends.

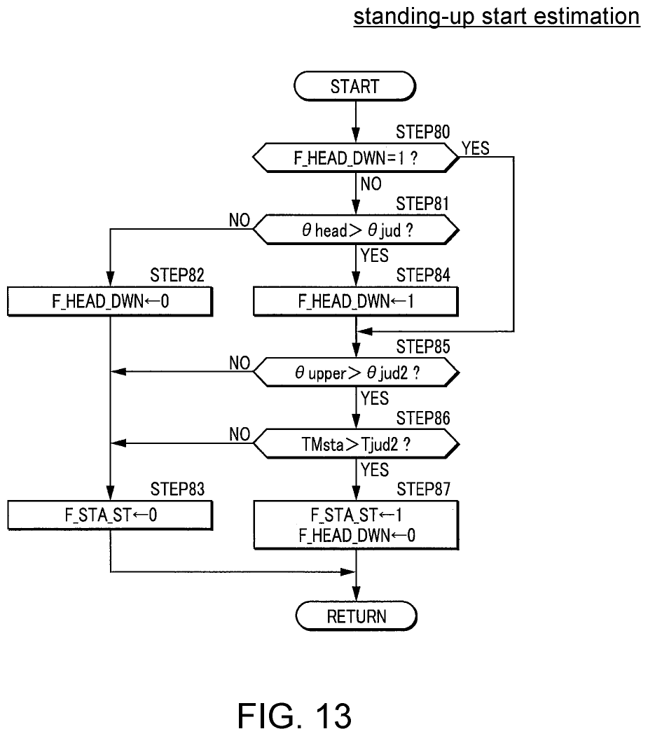

On the other hand, if the result of the determination is positive (FIG. 10/YES in STEP 44) and the user M is in a sitting state, a standing-up start estimation process is performed (FIG. 10/STEP 45). The standing-up start estimation process is to estimate whether the user M who is in the sitting state has started a standing-up motion and is performed specifically as shown in FIG. 13.

First, whether the head part forward tilt flag F_HEAD_DWN is “1” is determined (FIG. 13/STEP 80) as shown in the drawing.

If the result of the determination is negative (FIG. 13/NO in STEP 80), whether θhead>θjud is satisfied is determined (FIG. 13/STEP 81). If the result of the determination is negative (FIG. 13/NO in STEP 81) and the user M is not looking down in the forward direction, the head part forward tilt flag F_HEAD_DWN is set to “0” (FIG. 13/STEP 82).

Next, the user M is estimated not to have started a standing-up motion, a standing-up start flag F_STA_ST is set to “0” to indicate the state (FIG. 13/STEP 83). Then, the present process ends.

On the other hand, if the result of the determination is positive (FIG. 13/YES in STEP 81) and θhead>θjud is satisfied, the user M is estimated to be looking down in the forward direction and the head part forward tilt flag F_HEAD_DWN is set to “1” to indicate the state (FIG. 13/STEP 84).

If the head part forward tilt flag F_HEAD_DWN is set to “1” as described above or if the result of the determination is positive (FIG. 13/YES in STEP 80) and the head part forward tilt flag F_HEAD_DWN is set to “1” at the timing before the previous timing, whether θupper>θjud2 is satisfied is determined (FIG. 13/STEP 85).

θupper represents a forward tilt angle of the upper body of the user M and is calculated on the basis of detection signals of the waist motion sensor 28 and the head motion sensor 29. In addition, θjud2 represents a predetermined determination value for determining whether the user M has started a standing-up motion. Further, in the present embodiment, the forward tilt angle of the upper body θupper corresponds to a forward tilt state parameter.

If the result of the determination is negative (FIG. 13/NO in STEP 85), the standing-up start flag F_STA_ST is set to “0” as described above (FIG. 13/STEP 83), and then the present process ends.

On the other hand, if the result of the determination is positive (FIG. 13/YES in STEP 85), that is, if the user M is estimated to have his or her upper body tilt forward, whether TMsta>Tjud2 is satisfied is determined (FIG. 13/STEP 86). TMsta represents a time elapsed in a state in which θupper>θjud2 is satisfied, and Tjud2 represents a predetermined determination value for determining whether the user M is actually tilting his or her upper body forward.

If the result of the determination is negative (FIG. 13/NO in STEP 86), the standing-up start flag F_STA_ST is set to “0” as described above (FIG. 13/STEP 83), and then the present process ends.

On the other hand, if the result of the determination is positive (FIG. 13/YES in STEP 86), the user M is estimated to have started a standing-up motion, the standing-up start flag F_STA_ST is set to “1” to indicate the state, and at the same time, the head part forward tilt flag F_HEAD_DWN is reset to “0” (FIG. 13/STEP 87). Then, the present process ends.

Returning to FIG. 10, after the standing-up start estimation process (FIG. 10/STEP 45) is performed as described above, the motion start estimation process ends. In the motion start estimation process of FIG. 10, a start of a walking motion, a start of a standing-up motion, a start of a crouching motion, and the like are estimated as described above.

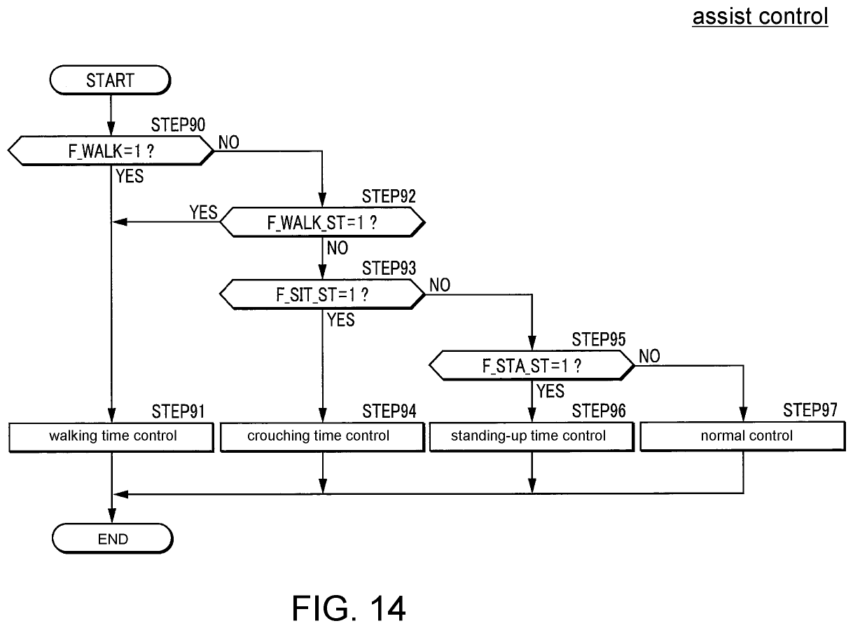

Next, an assist control process will be described with reference to FIG. 14. The assist control process is to control the walking assist device 1 according to a motion state of the user M and is performed by the assist controller 11 on a predetermined control cycle.

First, whether the above-described walking flag F_WALK is “1” is determined (FIG. 14/STEP 90) as shown in the drawing. If the result of the determination is positive (FIG. 14/YES in STEP 90) and the user M is walking, a walking time control process is performed (FIG. 14/STEP 91).

In the walking time control process, the drive device 9 is controlled such that an assisting force for helping and/or supporting a walking motion of the user M is generated in accordance with detection signals of the above-described various sensors 20 to 29. After the walking time control process is performed as described above, the present process ends.

On the other hand, if the result of the above-described determination is negative (FIG. 14/NO in STEP 90) and the user M is not walking, whether the above-described walking start flag F_WALK_ST is “1” is determined (FIG. 14/STEP 92).

If the result of the determination is positive (FIG. 14/YES in STEP 92) and the user M has started a walking motion, the walking time control process is performed as described above (FIG. 14/STEP 91), and then the present process ends.

On the other hand, if the result of the above-described determination is negative (FIG. 14/NO in STEP 92), whether the above-described crouching start flag F_SIT_ST is “1” is determined (FIG. 14/STEP 93). If the result of the determination is positive (FIG. 14/YES in STEP 93) and the user M has started a crouching motion, a crouching time control process is performed (FIG. 14/STEP 94).

In the crouching time control process, the drive device 9 is controlled such that an assisting force for helping and/or supporting a crouching motion of the user M is generated in accordance with detection signals of the above-described various sensors 20 to 29. After the crouching time control process is performed as described above, the present process ends.

On the other hand, if the result of the above-described determination is negative (FIG. 14/NO in STEP 93), whether the above-described standing-up start flag F_STA_ST is “1” is determined (FIG. 14/STEP 95). If the result of the determination is positive (FIG. 14/YES in STEP 95) and the user M has started a standing-up motion, a standing-up time control process is performed (FIG. 14/STEP 96).

In the standing-up time control process, the drive device 9 is controlled such that an assisting force for helping and/or supporting a standing-up motion of the user M is generated in accordance with detection signals of the above-described various sensors 20 to 29. After the standing-up time control process is performed as described above, the present process ends.

On the other hand, if the result of the above-described determination is negative (FIG. 14/NO in STEP 95), a normal control process is performed (FIG. 14/STEP 97). In this normal control process, when it is necessary to help and/or support a motion of the user M, the drive device 9 is controlled such that an assisting force is generated in accordance with detection signals of the above-described various sensors 20 to 29. After the normal control process is performed as described above, the present process ends.

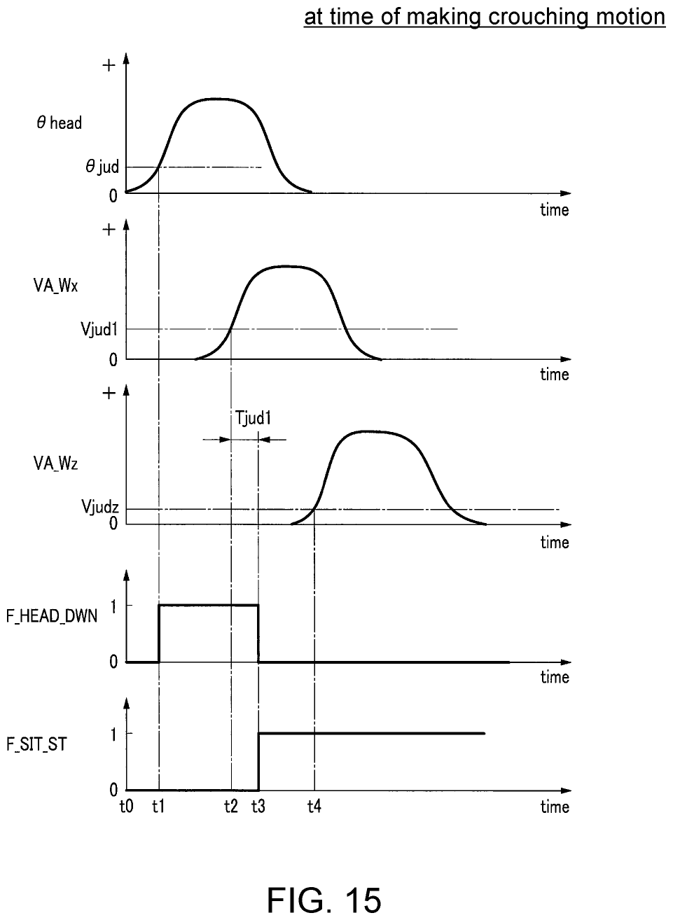

Next, transition of each parameter when the user M performs a crouching motion from a standing state and the like will be described with reference to FIG. 15. Further, VA_Wz in the drawing represents the absolute value of a z-axis speed of the waist part of the user M, and Vjusz represents a predetermined determination value for determining whether the user M has actually started to sit down.

As the user M starts the crouching motion at time t1, the forward tilt angle of the head part of the user M 0head starts increasing as illustrated in the drawing. Then, the head part forward tilt flag F_HEAD_ DWN is set to “1” at the timing at which θhead>θjud is satisfied (time t1).

Then, when the user M moves his or her waist part backward, VA_Wx>Vjud1 is satisfied (time t2). Then, when the user M is estimated to have started the crouching motion at the timing when a time corresponding to a determination value Tjud1 has elapsed from the timing at which VA_Wx>Vjud1 was satisfied (time t3), the crouching start flag F_SIT_ST is set to “1,” and at the same time, the head part forward tilt flag F_HEAD_DWN is set to “0.”

Accordingly, the crouching motion of the user M is helped and/or supported due to the crouching time control process performed from the time t3. In this case, for example, if the walking assist device 1 starts control at the timing at which VA_Wz>Vjudz is satisfied and the user M actually starts lowering his or her waist part (time t4), the walking assist device 1 is likely to obstruct the crouching motion of the user M until the walking assist device 1 actually generates an assisting force.

In order to solve this problem, according to the control device 10 of the present embodiment, since the crouching time control process is performed at an earlier timing (time t3) than the timing at which the user M actually starts lowering his or her waist part (time t4), it is ascertained that the crouching motion of the user M can be appropriately supported and/or helped without the above-described problem.

According to the control device 10 of the present embodiment, whether the user M is in a standing state is estimated and whether the user M is in a sitting state is estimated in accordance with detection signals of the left and right foot motion sensors 26 and 27 and the waist motion sensor 28 as described above. In this case, whether the user M is in a standing state can be accurately estimated and whether the user M is in a sitting state can also be accurately estimated using positional relations of the left and right sole parts with the waist part and with a height of the waist part.

In addition, the x-axis speed V_Wx and the absolute value VA_Wx of the waist part are calculated in accordance with a detection signal of the third motion sensor 28, and the forward tilt angle of the head part 0head is calculated in accordance with a detection signal of the fourth motion sensor 29. In addition, if both θhead>θjud, and V_Wx<0 and VA_Wx>Vjud1 are satisfied in a case where the user M is estimated to be in a standing state, the user M is estimated to have started a crouching motion from the standing state.

In this case, when a human starts a crouching motion from a standing state, a looking-down motion of the head part and a motion of the waist part moving backward are performed first as described above, and thus whether the user has started a crouching motion from a standing state can be accurately estimated assuming that the above-described conditions (θhead>θjud, V_Wx<0, and VA_Wx>Vjud1) are satisfied.

Then, in a case where the user M is estimated to have started the crouching motion, the walking assist device 1 is controlled such that the crouching motion is supported, and thus the crouching motion of the user M can be quickly and appropriately supported by the walking assist device 1.

In addition, if both V_Wx>Vjud2 and VA_Wy>Vjud3 are satisfied in a case where the user M is estimated to be in a standing state, the user M is estimated to have started a walking motion from the standing state. Since a movement of the waist part in a lateral direction is performed first in addition to a movement of the waist part forward when a human starts a walking motion from a standing state as described above, whether the user M has started the walking motion from the standing state can be accurately estimated assuming that the above-described conditions (V_Wx>Vjud2 and VA_Wy>Vjud3) are satisfied.

Then, in a case where the user M is estimated to have started the walking motion, the walking assist device 1 is controlled such that the walking motion is supported, and thus the walking motion of the user M can be quickly and appropriately supported by the walking assist device 1.

Furthermore, if both θhead>θjud and θupper>θjud2 are satisfied in a case where a forward tilt angle of the upper body θupper is calculated and the user M is estimated to be in a sitting state in accordance with detection signals of the third motion sensor 28 and the fourth motion sensor 29, the user M is estimated to have started a standing-up motion from the sitting state.

In this case, since a looking-down motion of the head part and a forward tilting motion of the upper body are performed first when a human starts a standing-up motion from a sitting state as described above, whether the user M has started the standing-up motion from the sitting state can be accurately estimated assuming that the above-described conditions (θhead>θjud and θupper>θjud2) are satisfied.

Then, in a case where the user M is estimated to have started the standing-up motion, the walking assist device 1 is controlled such that the standing-up motion is supported, and thus the standing-up motion of the user M can be quickly and appropriately supported by the walking assist device 1.

Further, in the crouching start estimation process of the embodiment in FIG. 11, the processes of STEPS 50 to 52 and 54 may be omitted, and the determination value Vjud1 of STEP 56 may be set to a greater value than that of FIG. 11. The reason for this operation is that, since there are cases of transition from the standing posture A1 to the forward tilt posture A3 with the head part of the user M in the looking-down posture A2 tilting at a smaller angle, a start of a crouching motion can be accurately estimated even if determination of whether a posture has changed from the standing posture A1 to the looking-down posture A2 is omitted.

Furthermore, the crouching start estimation process of the embodiment in FIG. 11, whether the tilt angle of the upper body of the user M exceeds a predetermined value may be determined or whether a tilt angle speed of the upper body of the user M exceeds a predetermined value may be determined instead of performing the determination processes of STEP 55 and 56, and if the result of the determination is positive, the process of STEP 57 may be performed. Even with the above-described configuration, a start of a crouching motion can be accurately estimated.

On the other hand, in the walking start estimation process of the embodiment in FIG. 12, the process of STEP 70 may be omitted, and the determination value Vjud3 of STEP 71 may be set to a greater value than that of FIG. 12. The reason for this operation is that, since there are cases where the user M has a small forward movement amount when he or she transitions from the standing posture C1 to the side tilt posture C2, a start of a walking motion can be accurately estimated even if determination of whether the user M has moved forward is omitted.

In addition, in the standing-up start estimation process of the embodiment in FIG. 13, the processes of STEPS 80 to 82 and 84 may be omitted, and the determination value θjud2 of STEP 85 may be set to a greater value than that of FIG. 13. The reason for this operation is that, since there are cases of transition from the sitting posture B1 to the forward tilt posture B3 with the head part of the user M in the looking-down posture B2 tilting at a smaller angle, a start of a standing-up motion can be accurately estimated even if determination of whether a posture has changed from the sitting posture B1 to the forward tilt posture B3 is omitted.

On the other hand, although the embodiment includes an example in which the x-axis speed of the waist part V_Wx, the absolute value of the x-axis speed of the waist part VA_Wx, and the absolute value of the y-axis speed of the waist part VA_Wy are used as waist movement state parameters, waist movement state parameters of one or some exemplary embodiments of the disclosure are not limited thereto, and any value indicating a movement state of the waist part of the user may be used. For example, an x-axis acceleration and a y-axis speed of the waist part, and the absolute values thereof may be used as waist movement state parameters.

In addition, although the embodiment includes an example in which the forward tilt angle θupper of the upper body is used as a forward tilt state parameter, a forward tilt state parameter of one or some exemplary embodiments of the disclosure is not limited thereto, and any value indicating a forward tilt state of the upper body of the user M may be used. For example, a forward tilt angular speed (or forward tilt angular acceleration) of the upper body may be used as a forward tilt state parameter, and in this case, whether a forward tilt angular speed (or forward tilt angular acceleration) of the upper body of the user M exceeds a predetermined value may be determined, instead of performing the determination process of STEP 85 in the standing-up start estimation process of the embodiment in FIG. 13.

Furthermore, a positional relation of a center position of the head part in the upper body of the user M with a center position of the waist part of the user M may be used as a forward tilt state parameter, and in this case, whether the center position of the head part in the upper body is positioned forward from the center position of the waist part by a predetermined value may be determined, instead of performing the determination process of STEP 85 in the standing-up start estimation process of the embodiment in FIG. 13.

In addition, although the embodiment includes an example in which the active-type walking assist device 1 is used as a motion support device, a motion support device of one or some exemplary embodiments of the disclosure is not limited thereto, and any device that supports motions of at least the lower body of a human is possible. For example, an active-type assist device that supports motions of the upper body as well as the lower body of a human may be used as a motion support device. Furthermore, a passive-type walking assist device without a power source may be used as a motion support device.

On the other hand, although the embodiment includes an example in which the left foot motion sensor 26 is used as the first motion sensor, the first motion sensor of one or some exemplary embodiments of the disclosure is not limited thereto, and a sensor that detects motions of the left sole part may be used. For example, an acceleration sensor, a gyro sensor, or the like may be used as the first motion sensor. In addition, the left foot motion sensor 26 may be mounted direct1y on the left sole part of the user.

In addition, although the embodiment includes an example in which the right foot motion sensor 27 is used as the second motion sensor, the second motion sensor of one or some exemplary embodiments of the disclosure is not limited thereto, and a sensor that detects motions of the right sole part may be used. For example, an acceleration sensor, a gyro sensor, or the like may be used as the second motion sensor. In addition, the right foot motion sensor 27 may be mounted direct1y on the right sole part of the user.

In addition, although the embodiment includes an example in which the waist motion sensor 28 is used as the third motion sensor, the third motion sensor of one or some exemplary embodiments of the disclosure is not limited thereto, and a sensor that detects motions of the waist part may be used. For example, an acceleration sensor, a gyro sensor, or the like may be used as the third motion sensor. In addition, the waist motion sensor 28 may be provided in the seat member 2 of the walking assist device 1.

On the other hand, although the embodiment includes an example in which the head motion sensor 29 is used as the fourth motion sensor, the fourth motion sensor of one or some exemplary embodiments of the disclosure is not limited thereto, and a sensor that detects motions of the head part may be used. For example, an acceleration sensor, a gyro sensor, or the like may be used as the fourth motion sensor.

What is claimed is:

1. A control device of a motion support device that is worn by a user to support a motion of at least a lower body of the user, the control device comprising:
a first motion sensor capable of detecting a motion of a left sole part of the user;
a second motion sensor capable of detecting a motion of a right sole part of the user;
a third motion sensor capable of detecting a motion of a waist part of the user;
a standing state estimation part estimating whether the user is in a standing state in accordance with a detection signal of the first to third motion sensors;
a waist movement state parameter calculation part calculating a waist movement state parameter indicating a movement state of the waist part of the user in accordance with the detection signal of the third motion sensor;
a motion estimation part estimating that the user has started a predetermined motion from the standing state if the waist movement state parameter has a value in a predetermined range indicating a movement of the waist part of the user in one of a backward direction and a lateral direction in a case where the user is estimated to be in the standing state; and
a control part controlling the motion support device such that the predetermined motion is supported in a case where the user is estimated to have started the predetermined motion.

2. The control device of the motion support device according to claim 1,
wherein the motion estimation part estimates that the user has started a crouching motion as the predetermined motion from the standing state if the waist movement state parameter has a value in the predetermined range indicating a movement of the waist part of the user in the backward direction, and
wherein the control part controls the motion support device such that the crouching motion is supported in a case where the user is estimated to have started the crouching motion from the standing state.

3. The control device of the motion support device according to claim 2, the control device further comprising:
a fourth motion sensor capable of detecting a motion of a head part of the user; and
a forward tilt angle calculation part calculating a forward tilt angle of the head part of the user in accordance with a detection signal of the fourth motion sensor,
wherein the motion estimation part estimates that the user has started the crouching motion from the standing state if the waist movement state parameter has a value in the predetermined range and the forward tilt angle of the head part of the user has a value in a second predetermined range in the case where the user is estimated to be in the standing state.

4. The control device of the motion support device according to claim 1,
wherein the motion estimation part estimates that the user has started a walking motion as the predetermined motion from a standing state if the waist movement state parameter has a value in the predetermined range indicating a movement of the waist part in the lateral direction in a case where the user is estimated to be in the standing state, and
wherein the control part controls the motion support device such that the walking motion is supported in a case where the user is estimated to have started the walking motion from the standing state.

5. The control device of the motion support device according to claim 4,
wherein the motion estimation part estimates that the user has started the walking motion from the standing state if the waist movement state parameter has a value in the predetermined range indicating the movement of the waist part in the lateral direction and a value in a third predetermined range indicating a movement of the waist part in a forward direction in the case where the user is estimated to be in the standing state.

6. A control device of a motion support device that is worn by a user to support a motion of at least a lower body of the user, the control device comprising:
a first motion sensor capable of detecting a motion of a left sole part of the user;
a second motion sensor capable of detecting a motion of a right sole part of the user;
a third motion sensor capable of detecting a motion of a waist part of the user;
a fourth motion sensor capable of detecting a motion of a head part of the user;
a sitting state estimation part estimating whether the user is in a sitting state in accordance with a detection signal of the first to third motion sensors;
a forward tilt state parameter calculation part calculating a forward tilt state parameter indicating a forward tilt state of an upper body of the user in accordance with the detection signal of the third motion sensor and the fourth motion sensor;
a motion estimation part estimating that the user has started a standing-up motion from the sitting state if the forward tilt state parameter has a value in a fourth predetermined range indicating a forward tilt of the upper body of the user in a case where the user is estimated to be in the sitting state; and
a control part controlling the motion support device such that the standing-up motion is supported in a case where the user is estimated to have started the standing-up motion.

7. The control device of the motion support device according to claim 6, the control device further comprising:
a forward tilt angle calculation part calculating a forward tilt angle of the head part of the user in accordance with the detection signal of the fourth motion sensor,
wherein the motion estimation part estimates that the user has started the standing-up motion from the sitting state if the forward tilt state parameter has the value in the fourth predetermined range and the forward tilt angle of the head part has a value in a fifth predetermined range in the case where the user is estimated to be in the sitting state.
